(19) United States
(12) Patent Application Publication
(10) Pub. No.: US 2022/0047563 A1
(43) Pub. Date: Feb. 17, 2022

(54) COMBINATION THERAPY FOR THE TREATMENT OF ESTROGEN-RECEPTOR POSITIVE BREAST CANCER

(71) Applicant: ZENITH EPIGENETICS LTD., Calgary (CA)

(72) Inventors: Eric CAMPEAU, Calgary (CA); Olesya KHARENKO, Calgary (CA); Edward T.H. VAN DER HORST, Palo Alto, CA (US)

(21) Appl. No.: 17/275,462

(22) PCT Filed: Sep. 13, 2019
(86) PCT No.: PCT/IB2019/001035

§ 371 Date: Mar. 11, 2021

Related U.S. Application Data
(60) Provisional application No. 62/730,837, filed on Sep. 13, 2018.

(51) Int. Cl. A61K 31/437; A61K 31/506; A61K 31/565; A61K 31/519; A61P 35/00

(52) U.S. Cl. CPC A61K 31/437; A61K 31/506; A61P 35/00; A61K 31/519; A61K 31/565

(57) ABSTRACT

Methods for treating estrogen receptor positive (ER+) breast cancer, comprising administering to a subject in need thereof, a BET bromodomain inhibitor in combination with a second agent, selected from a selective-estrogen receptor degrader (SERD), a selective-estrogen receptor modulator (SERM) and a selective CDK4/6 inhibitor. The BET bromodomain inhibitor is selected from 1-benzyl-6-(3,5-dimethylisoxazol-4-yl)-N-methyl-1H-imidazo[4,5-b]pyridin-2-amine (Compound I), 1-benzyl-6-(3,5-dimethylisoxazol-4-yl)-1H-imidazo [4,5-b]pyridin-2-amine, and pharmaceutically acceptable salts/co-crystals thereof.

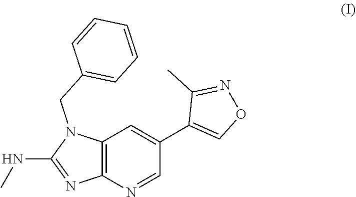 [SMILES: CNc1nc2ncc(-c3conc3C)cc2n1Cc1ccccc1]

### BACKGROUND

More than 200,000 women are diagnosed with breast cancer every year in the United States. About 80% of these cases are estrogen receptor positive (ER+), which is characterized by the up-regulation of ER signaling. Current lines of therapies include endocrine therapies which have resulted in great treatment improvements in ER+breast cancer. Unfortunately, resistance to these therapies occurs over time and development of additional therapeutic strategies is needed. Recently, the bromodomain and extra terminal (BET) proteins BRD3 and BRD4 were shown to be involved in the transcription of ER (Feng et al., 2014). Treatment with some BET inhibitor can suppress ER-mediated signaling, offering a potential strategy to overcome endocrine resistance by further ER-signaling suppression regardless of ESR1 mutation status (Feng et al., 2014; Ladd et al., 2016; Nagarajan et al., 2014; Sengupta et al., 2015). However, it remains unclear if BRD3 and/or BRD4 are involved in the resistance mechanisms to endocrine therapies in patients, and whether BET inhibitor can potently inhibit the proliferation of ER+ breast cancer cells that are resistant to CDK4/6 inhibitors. CDK4/6 inhibitors are standard of care in first and second line metastatic ER+ breast cancer, and a combination of a CDK4/6 inhibitor and a BET inhibitor can be next line of therapy for subjects developing resistance to the CDK4/6 mono therapy.

However, at this time, it is unclear which, if any, BET inhibitors will result in clinical benefit when administered to subjects with ER+ breast cancer. It is also unclear which, if any BET inhibitors will combine synergistically with other drugs, such as an a selective-estrogen receptor degrader (SERD), a selective-estrogen receptor modulator (SERM), an aromatase inhibitor (AI), or a selective CDK4/6 inhibitor, in the treatment of breast cancer; what level of synergy is required; and which second therapeutic agent will be the best combination partner for each BET inhibitor, resulting in clinical benefit when administered to patients with breast cancer. In addition to a clinical benefit, the combination also has to be safe and well tolerated at the efficacious doses. At this time, it cannot be predicted which combination will show the best overall profile.

### SUMMARY

The present invention discloses methods of treating estrogen receptor positive (ER+) breast cancer by concomitant administration of a BET bromodomain inhibitor, or a pharmaceutically acceptable salt or co-crystal of a BET bromodomain inhibitor, and a second therapeutic agent to a subject in need thereof.

In some embodiments of the invention, the method of treating ER+ breast cancer is a triple combination therapy comprising administration of a BET bromodomain inhibitor, a second therapeutic agent, and an estrogen receptor modulator.

In some embodiments, the BET bromodomain inhibitor is administered simultaneously with the second therapeutic agent and optionally with the estrogen receptor modulator. In some embodiments, the BET bromodomain inhibitor is administered sequentially with the second therapeutic agent and optionally with the estrogen receptor modulator. In some embodiments, the BET bromodomain inhibitor is administered in a single pharmaceutical composition with the second therapeutic agent and optionally with the estrogen receptor modulator. In some embodiments, the BET bromodomain inhibitor and the second therapeutic agent and optionally the estrogen receptor modulator are administered as separate compositions. In some embodiments, the BET bromodomain inhibitor and the second therapeutic agent are in a single composition and the optional estrogen receptor modulator is in a separate composition.

In some embodiments, the second therapeutic agent is an agent used in the treatment of breast cancer.

In some embodiments, the second therapeutic agent is a selective-estrogen receptor degrader (SERD) or modulator (SERM).

In some embodiments, the second therapeutic agent is a selective CDK4/6 inhibitor.

In some embodiments, the BET bromodomain inhibitor is a compound of Formula Ia or Formula Ib

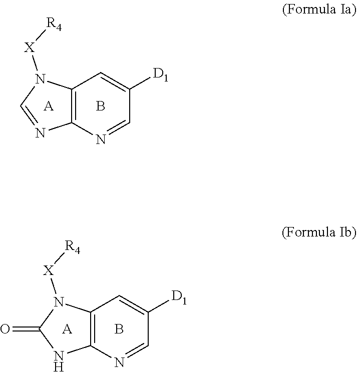

or a stereoisomer, tautomer, pharmaceutically acceptable salt, or co-crystal thereof, wherein:
Ring A and Ring B may be optionally substituted with groups independently selected from hydrogen, deuterium, —NH₂, amino, heterocycle(C₄-C₆), carbocycle(C₄-C₆), halogen, —CN, —OH, —CF₃, alkyl (C₁-C₆), thioalkyl (C₁-C₆), alkenyl (C₁-C₆), and alkoxy (C₁-C₆);
X is selected from —NH—, —CH₂—, —CH₂CH₂—, —CH₂CH₂CH₂—, —CH₂CH₂O—, —CH₂CH₂NH—, —CH₂CH₂S—,
   —C(O)—, —C(O)CH₂—, —C(O)CH₂CH₂—, —CH₂C(O)—, —CH₂CH₂C(O)—, —C(O)NH—, —C(O)O—, —C(O)S—, —C(O)NHCH₂—,
   —C(O)OCH₂—, —C(O)SCH₂—, wherein one or more hydrogen may independently be replaced with deuterium, hydroxyl, methyl, halogen, —CF₃, ketone, and where S may be oxidized to sulfoxide or sulfone;
R₄ is selected from optionally substituted 3-7 membered carbocycles and heterocycles; and
D₁ is selected from the following 5-membered monocyclic heterocycles:

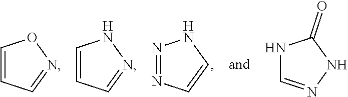

which are optionally substituted with hydrogen, deuterium, alkyl (C₁-C₄), alkoxy (C₁-C₄), amino, halogen, amide, —CF₃, —CN, —N₃, ketone (C₁-C₄), —S(O)Alkyl(Ci-C₄), —SO₂alkyl(C₁-C₄), -thioalkyl(C₁-C₄), —COOH, and/or ester, each of which may be optionally substituted with hydrogen, F, Cl, Br, —OH, —NH₂, —NHMe, —OMe, —SMe, oxo, and/or thio-oxo.

In some embodiments, the BET bromodomain inhibitor is a compound of Formula la. In some embodiments the compound of Formula Ia is 1-benzyl-6-(3,5-dimethylisoxazol-4-yl)-N-methyl-1H-imidazo[4,5-b]pyridine-2-amine (Compound I), which has the following formula:

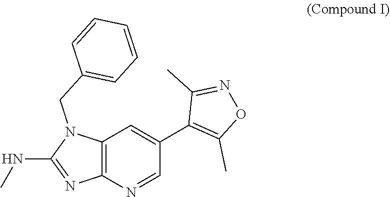 [SMILES: CNc1nc2ncc(-c3c(C)noc3C)cc2n1Cc1ccccc1]

In some embodiments, the BET bromodomain inhibitor is a pharmaceutically acceptable salt or co-crystal of a compound of Formula Ia or Formula Ib. In some embodiments, the BET bromodomain inhibitor is a pharmaceutically acceptable salt or co-crystal of Compound I. In some embodiments, the BET bromodomain inhibitor is a mesylate salt/co-crystal of Compound I Form I.

### BRIEF DESCRIPTION OF THE DRAWINGS

FIG. 1 Synergetic combination of the Compound I with abemaciclib in WT-MCF7 cells. CI=0.08

FIG. 2 Synergetic combination of the Compound I with abemaciclib in Palbo-R-MCF7 cells. CI=0.20

FIG. 3 Synergetic combination of the Compound I with abemaciclib in ZR-75-1 cells. CI=0.14

FIG. 4 Synergetic combination of the Compound I with abemaciclib in Palbo-R ZR-75-1 cells. CI=0.35

FIG. 5 Synergetic combination of the Compound I with abemaciclib in Abema-R MCF7 cells. CI=0.30

FIG. 6 Synergetic combination of the Compound I with fulvestrant in WT-MCF7 cells. CI=0.51.

FIG. 7 shows an X-ray powder diffractogram (XRPD) of a mesylate salt/co-crystal of Compound I.

FIG. 8 shows a differential scanning calorimeter (DSC) curve of a mesylate salt/co-crystal of Compound I.

FIG. 9 shows a thermogravimetric analysis (TGA) of a mesylate salt/co-crystal of Compound I.

### DEFINITIONS

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “hydrate” refers to a crystal form with either a stoichiometric or non-stoichiometric amount of water is incorporated into the crystal structure.

The term “alkenyl” as used herein refers to an unsaturated straight or branched hydrocarbon having at least one carbon-carbon double bond, such as a straight or branched group of 2-8 carbon atoms, referred to herein as (C₂-C₈) alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-8 carbon atoms, referred to herein as (C₁-C₈) alkoxy. Exemplary alkoxy groups include, but are not limited to, methoxy and ethoxy.

The term “alkyl” as used herein refers to a saturated straight or branched hydrocarbon, such as a straight or branched group of 1-8 carbon atoms, referred to herein as (C₁-C₈) alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “amide” as used herein refers to —NRₐC(O)(R_{b})— or —C(O)NR_{b}R_{c}, wherein Rₐ, R_{b} and R_{c} are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{b}, or R_{c}. The amide also may be cyclic, for example R_{b} and R_{c}, may be joined to form a 3- to 8-membered ring, such as 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., -amide-COOH or salts such as -amide-COONa, an amino group attached to a carboxy group (e.g., -amino-COOH or salts such as -amino-COONa).

The term “amine” or “amino” as used herein refers to the form —NR_{d}Rₑ or —N(R_{d})Rₑ—, where R_{d} and Rₑ are independently selected from alkyl, alkenyl, alkynyl, aryl, arylalkyl, carbamate, cycloalkyl, haloalkyl, heteroaryl, heterocycle, and hydrogen. The amino can be attached to the parent molecular group through the nitrogen. The amino also may be cyclic, for example any two of R_{d} and Rₑ may be joined together or with the N to form a 3- to 12-membered ring (e.g., morpholino or piperidinyl). The term amino also includes the corresponding quaternary ammonium salt of any amino group. Exemplary amino groups include alkylamino groups, wherein at least one of R_{d} or Rₑ is an alkyl group. In some embodiments Rd and Re each may be optionally substituted with hydroxyl, halogen, alkoxy, ester, or amino.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to, a monocyclic aromatic ring system, wherein the ring comprises 6 carbon atoms, referred to herein as “(C₆) aryl.”

The term “arylalkyl” as used herein refers to an alkyl group having at least one aryl substituent (e.g., -aryl-alkyl-). Exemplary arylalkyl groups include, but are not limited to, arylalkyls having a monocyclic aromatic ring system, wherein the ring comprises 6 carbon atoms, referred to herein as “(C₆) arylalkyl.”

The term “carbamate” as used herein refers to the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of R_{g}, Rₕ and Rᵢ are independently selected from aryl or heteroaryl, such as pyridine, pyridazine, pyrimidine, and pyrazine).

The term “carbocycle” as used herein refers to an aryl or cycloalkyl group.

The term “carboxy” as used herein refers to —COOH or its corresponding carboxylate salts (e.g., —COONa). The term carboxy also includes “carboxycarbonyl,” e.g. a carboxy group attached to a carbonyl group, e.g., —C(O)—COOH or salts, such as —C(O)—COONa.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃-C₈) cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure —C(O)O—, —C(O)O—Rⱼ₋, —RₖC(O)O—Rⱼ₋, or —RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rₖ can be a hydrogen, but Rⱼ cannot be hydrogen. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rj or Rk is alkyl, such as —O—C(O)-alkyl, —C(O)—O-alkyl-, and -alkyl-C(O)—O-alkyl-. Exemplary esters also include aryl or heteoraryl esters, e.g. wherein at least one of Rj or Rk is a heteroaryl group such as pyridine, pyridazine, pyrimidine and pyrazine, such as a nicotinate ester. Exemplary esters also include reverse esters having the structure —RₖC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The terms “halo” or “halogen” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅) heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)-Rn (such as acetyl, —C(O)CH₃) or —Rₙ₋C(O)—Rₒ₋. The ketone can be attached to another group through Rₙ or Rₒ. Rₙ or Rₒ can be alkyl, alkenyl, alkynyl, cycloalkyl, heterocyclyl or aryl, or Rₙ or Rₒ can be joined to form a 3- to 12-membered ring.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide and thioketone.

The term “thioalkyl” as used herein refers to an alkyl group attached to a sulfur (—S-alkyl-).

“Alkyl,” “alkenyl,” “alkynyl”, “alkoxy”, “amino” and “amide” groups can be optionally substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, thioketone, ureido and N. The substituents may be branched to form a substituted or unsubstituted heterocycle or cycloalkyl.

As used herein, a suitable substitution on an optionally substituted substituent refers to a group that does not nullify the synthetic or pharmaceutical utility of the compounds of the present disclosure or the intermediates useful for preparing them. Examples of suitable substitutions include, but are not limited to: C₁₋₈ alkyl, alkenyl or alkynyl; C₁₋₆ aryl, C₂₋₅ heteroaryl; C₃₇ cycloalkyl; C₁₋₈ alkoxy; C₆ aryloxy; —CN; —OH; oxo; halo, carboxy; amino, such as —NH(C₁₋₈ alkyl), —N(C₁₋₈ alkyl)₂, —NH((C₆)aryl), or —N((C₆)aryl)₂; formyl; ketones, such as —CO(C₁₋₈ alkyl), —CO((C₆aryl) esters, such as —CO₂(C₁₋₈ alkyl) and —CO₂ (C₆aryl). One of skill in art can readily choose a suitable substitution based on the stability and pharmacological and synthetic activity of the compound of the present disclosure.

The term “pharmaceutically acceptable composition” as used herein refers to a composition comprising at least one compound as disclosed herein formulated together with one or more pharmaceutically acceptable carriers.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

### Exemplary Embodiments

As summarized above, the invention provides methods of treating ER+ breast cancer with a combination therapy comprising administration of a BET bromodomain inhibitor of Formula Ia or Formula Ib, or a stereo isomer, tautomer, or pharmaceutically acceptable salt/co-crystal thereof, and a second therapeutic agent to a subject in need thereof:

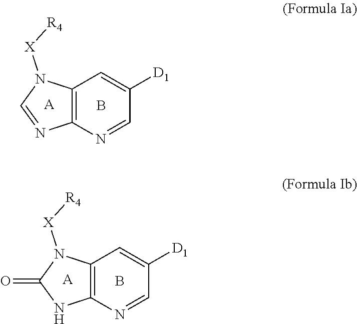

wherein:
Ring A and Ring B may be optionally substituted with groups independently selected from hydrogen, deuterium, —NH₂, amino, heterocycle(C₄-C₆), carbocycle(C₄-C₆), halogen, —CN, —OH, —CF₃, alkyl (C₁-C₆), thioalkyl (C₁-C₆), alkenyl (C₁-C₆), and alkoxy (C₁-C₆);
X is selected from —NH—, —CH₂—, —CH₂CH₂—, —CH₂CH₂CH₂—, —CH₂CH₂O—, —CH₂CH₂NH—, —CH₂CH₂S—, —C(O)—, —C(O)CH₂—, —C(O)CH₂CH₂—, —CH₂C(O)—, —CH₂CH₂C(O)—, —C(O)NH—, —C(O)O—, —C(O)S—, —C(O)NHCH₂—, —C(O)OCH₂—, —C(O)SCH₂—, wherein one or more hydrogen may independently be replaced with deuterium, hydroxyl, methyl, halogen, —CF₃, ketone, and where S may be oxidized to sulfoxide or sulfone;
R₄ is selected from optionally substituted 3-7 membered carbocycles and heterocycles; and
D₁ is selected from the following 5-membered monocyclic heterocycles:

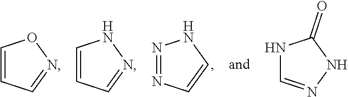

which are optionally substituted with hydrogen, deuterium, alkyl (C₁-C₄), alkoxy (C₁-C₄), amino, halogen, amide, —CF₃, —CN, —N₃, ketone (C₁-C₄), —S(O)Alkyl(C₁-C₄), —SO₂alkyl(C₁-C₄), -thioalkyl(C₁-C₄), —COOH, and/or ester, each of which may be optionally substituted with hydrogen, F, Cl, Br, —OH, —NH₂, —NHMe, —OMe, —SMe, oxo, and/or thio-oxo.

Compounds of Formula Ia and Ib, including Compound I, have been previously described in International Patent Publication WO 2015/002754, incorporated herein by reference in its entirety, and particularly for its description of the compounds of Formula Ia and Formula Ib, including Compound I, their synthesis, and the demonstration of their BET bromodomain inhibitor activity.

In some embodiments, the BET bromodomain inhibitor of Formula Ia and Formula Ib is selected from:

1-Benzyl-6-(3,5-dimethylisoxazol-4-yl)-N-ethyl-1H-imidazo[4,5-b]pyridin-2-amine;
1-Benzyl-6-(3,5-dimethylisoxazol-4-yl)-N-methyl-1H-imidazo[4,5-b]pyridin-2-amine;
N,1-Dibenzyl-6-(3,5-dimethylisoxazol-4-yl)-1H-imidazo[4,5-b]pyridin-2-amine;
1-Benzyl-6-(3,5-dimethylisoxazol-4-yl)-N-(pyridin-3-ylmethyl)-1H-imidazo[4,5-b]pyridin-2-amine;
4-(1-Benzyl-2-(pyrrolidin-1-yl)-1H-imidazo[4,5-b]pyridin-6-yl)-3,5-dimethylisoxazole;
4-(2-(Azetidin-1-yl)-1-(cyclopentylmethyl)-1H-imidazo[4,5-b]pyridin-6-yl)-3,5-dimethylisoxazole;
1-Benzyl-6-(3,5-dimethylisoxazol-4-yl)-1H-imidazo[4,5-b]pyridin-2-amine;
1-(cyclopentylmethyl)-6-(3,5-dimethylisoxazol-4-yl)-N-(tetrahydro-2H-pyran-4-yl)-1H-imidazo[4,5-b]pyridin-2-amine;
4-Amino-1-benzyl-6-(3,5-dimethylisoxazol-4-yl)-1H-benzo[d]imidazol-2(3H)-one;
4-Amino-6-(3,5-dimethylisoxazol-4-yl)-1-(4-methoxybenzyl)-1H-benzo[d]imidazol-2(3H)-one;
4-Amino-6-(3,5-dimethylisoxazol-4-yl)-1-(1-phenylethyl)-1H-benzo[d]imidazol-2(3H)-one;
4-Amino-1-benzyl-6-(3,5-dimethylisoxazol-4-yl)-3-methyl-1H-benzo[d]imidazol-2(3H)-one;
or a pharmaceutically acceptable salt or co-crystal thereof.

In one embodiment, the invention provides a method for treating ER+ breast cancer comprising administrating to a subject in need thereof, a compound selected from 1-benzyl-6-(3,5-dimethylisoxazol-4-yl)-N-methyl-1H-imidazo[4,5-b]pyridin-2-amine (Compound I) and 1-benzyl-6-(3,5-dimethylisoxazol-4-yl)-1H-imidazo[4,5-b]pyridin-2-amine and pharmaceutically acceptable salts or co-crystals thereof, concomitantly with another therapeutic agent.

In one embodiment, wherein the BET bromodomain inhibitor administered in the methods of the invention is the mesylate salt or co-crystal of 1-benzyl-6-(3,5-dimethylisoxazol-4-yl)-N-methyl-1H-imidazo[4,5-b]pyridin-2-amine (Compound I).

In one embodiment, a compound selected from 1-benzyl-6-(3,5-dimethylisoxazol-4-yl)-N-methyl-1H-imidazo[4,5-b]pyridin-2-amine (Compound I) and 1-benzyl-6-(3,5-dimethylisoxazol-4-yl)-1H-imidazo[4,5-b]pyridin-2-amine and pharmaceutically acceptable salts or co-crystals thereof, is dosed with a second therapeutic agent without resulting in thrombocytopenia as a dose-limiting toxicity.

In some embodiments, the second therapeutic agent administered in the methods of the invention is a selective-estrogen receptor degrader (SERD).

In some embodiments, the second therapeutic agent administered in the methods of the invention is a selective-estrogen receptor modulator (SERM).

In some embodiments, the second therapeutic agent is tamoxifen.

In some embodiments, the subject has previously been treated with an aromatase inhibitor.

In some embodiments, the second therapeutic agent is fulvestrant.

In some embodiments, the second therapeutic agent is a CDK4/6 inhibitor.

In some embodiments, the second therapeutic agent is selected from abemaciclib, ribociclib, and palbociclib.

In some embodiments, the second therapeutic agent is abemaciclib.

In some embodiments, the subject previously has been treated with a breast cancer therapy. In some embodiments, the prior breast cancer therapy is chemotherapy. In some embodiments, the prior breast cancer therapy is treatment with a CDK4/6 inhibitor. In some embodiments, the prior breast cancer therapy is immunotherapy.

In some embodiments, the subject is a human.

In some embodiments, the BET bromodomain inhibitor as described herein is administered concomitantly with the second therapeutic agent. “Concomitantly” as used herein means that the BET bromodomain inhibitor of Formula Ia or Formula Ib and the second therapeutic agent are administered with a time separation of a few seconds (for example 15 sec., 30 sec., 45 sec., 60 sec. or less), several minutes (for example 1 min., 2 min., 5 min. or less, 10 min. or less, 15 min. or less), or 1-12 hours. When administered concomitantly, the BET bromodomain inhibitor and the other therapeutic agent may be administered in two or more administrations, and contained in separate compositions or dosage forms, which may be contained in the same or different package or packages.

### LIST OF REFERENCES

Chou, T. C. and Talalay, P. (2984), Quantitative analysis of dose-effect relationships: The combined effects of multiple drugs or enzyme inhibitors. Advances in Enzyme Regulation 22, 27-55.
Feng, Q., Zhang, Z., Shea, M. J., Creighton, C. J., Coarfa, C., Hilsenbeck, S. G., Lanz, R., He, B., Wang, L., Fu, X., et al. (2014). An epigenomic approach to therapy for tamoxifen-resistant breast cancer. Cell Res 24, 809-819.
Ladd, B., Mazzola, A. M., Bihani, T., Lai, Z., Bradford, J., Collins, M., Barry, E., Goeppert, A.U., Weir, H. M., Hearne, K., et al. (2016). Effective combination therapies in preclinical endocrine resistant breast cancer models harboring ER mutations. Oncotarget 7, 54120-54136.
Nagarajan, S., Hossan, T., Alawi, M., Najafova, Z., Indenbirken, D., Bedi, U., Taipaleenmaki, H., Ben-Batalla, I., Scheller, M., Loges, S., et al. (2014). Bromodomain Protein BRD4 Is Required for Estrogen Receptor-Dependent Enhancer Activation and Gene Transcription. Cell reports 8, 460-469.
Sengupta, S., Biarnes, M., Clarke, R., and Jordan, V. C. (2015). Inhibition of BET proteins impairs estrogen-mediated growth and transcription in breast cancers by pausing RNA polymerase advancement. Breast Cancer Res Treat 150, 265-278.

### EXAMPLES

Tissue culture media and reagents were obtained from ThermoFisher Scientific. Fulvestrant was obtained from Sigma, abemaciclib and palbociclib were obtained from Selleckchem.

### Example 1: Synthesis of Compound 1

### Step A: Synthesis of 5-Bromo-N³-(phenylmethylene)pyridine-2,3-diamine (Compound B)

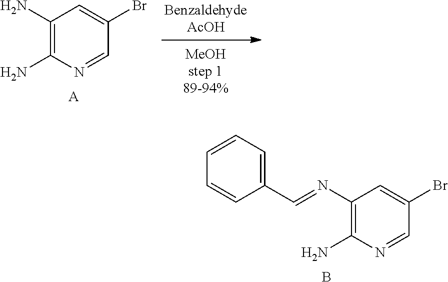

Starting material A was dissolved in methanol and acetic acid. The solution was cooled to 0-5° C. and benzaldehyde was added dropwise. Once the reaction was complete, process water and a NaHCO₃ solution was added dropwise, keeping the temperature low (0-5° C.). The solid was filtered off and washed with methanol/water 1:1, followed by drying, leaving Compound B in 94% yield and +99% purity by HPLC. ¹H-NMR (DMSO-d₆): δ8.75 (1H), 8.04 (2H), 7.93 (1H), 7.65 (1H), 7.50-7.60 (3H).

### Step B: Synthesis of N³-Benzyl-5-bromopyridine-2,3-diamine (Compound C)

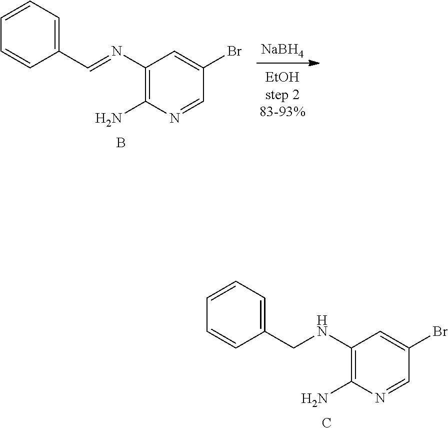

Compound B was dissolved in ethanol and NaHB₄ was added in portions keeping the temperature between 15-25° C. The reaction mixture was stirred for 8-15 h until the reaction was complete as monitored by HPLC. A HCl solution was added, adjusting pH to 6-7, followed by process water, keeping the temperature between 15-25° C. The mixture was stirred for 1-5 h, filtered and washed with an ethanol/water mixture. Following drying at ˜60° C. for 15-20 h, Compound C was obtained. ¹H-NMR (DMSO-d₆): d 7.2-7.4 (6 H), 6.55 (1 H), 5.70-5.83 (3 H), 4.30 (2 H).

### Step C: Synthesis of N³-Benzyl-5-(3,5-dimethyl-1,2-oxazol-4-yl)pyridine-2,3-diamine (Compound D)

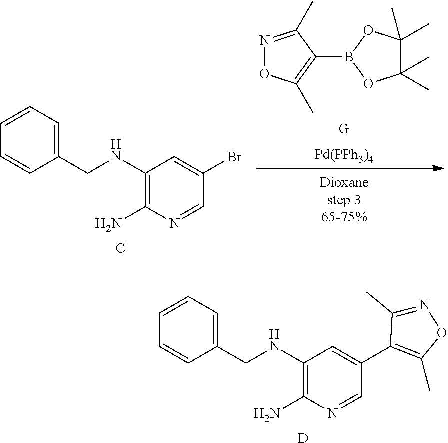

Compound C, Compound G, and potassium phosphate tribasic trihydrate were mixed followed by addition of 1,4-Dioxane and process water. The resulting mixture was thoroughly purged with nitrogen. Tetrakis(triphenylphosphine)palladium(0) was added and the mixture was heated to ≥90° C. until the ratio of Compound C to Compound D was not more than 1%. After cooling, the reaction mixture was filtered, the solid washed with 1,4-dioxane and then concentrated. Process water was added and the mixture was stirred until the amount of Compound D remaining in the mother liquors was not more than 0.5%. Compound D was isolated by filtration and sequentially washed with 1,4-dioxane/water and t-butylmethyl ether. The wet cake was mixed in methylene chloride and silica gel. After stirring, the mixture was filtered then concentrated. The mixture was cooled and t-butylmethyl ether was added. The product was isolated by filtration and dried until the methylene chloride, t-butylmethyl ether, and moisture levels are not more than 0.5%. ¹H-NMR (DMSO-d₆): δ7.30-7.45 (4 H), 7.20-7.25 (2 H), 6.35 (1 H), 5.65-5.80 (3 H), 4.30-4.40 (2 H), 2.15 (3 H), 1.95 (3 H).

### Step D: Synthesis of 1-Benzyl-6-(3,5-dimethyl-1,2-oxazol-4-yl)-3H-imidazo[4,5-b]pyridin-2-one (Compound E)

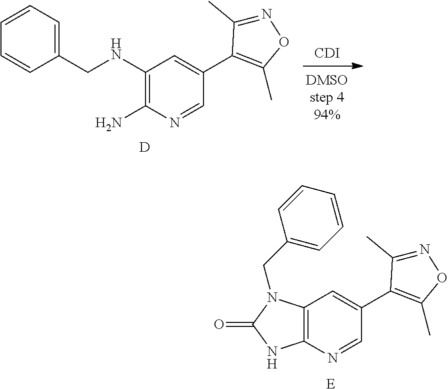

Carbonyldiimidazole solid was added to a stirring mixture of Compound D and dimethylsulfoxide. The mixture was heated until the ratio of Compound D to Compound E was NMT 0.5%. The mixture was cooled and process water was added over several hours. The resulting mixture was stirred at ambient temperature for at least 2 h. The product was isolated by filtration and washed with process water. The dimethylsulfoxide was verified to be NMT 0.5% before drying using heat and vacuum. Drying was complete when the moisture level was NMT 0.5%, leaving Compound E. ¹H-NMR (DMSO-ds): δ11.85 (1H), 7.90 (1H), 7.20-7.45 (6H), 5.05 (2H), 3.57 (3H), 2.35 (3H), 2.15 (3H).

### Step E: Synthesis of 4-[1-Benzyl-2-chloro-1H-imidazo[4,5-b]pyridine-6-yl]-3,5-dimethyl-1,2-oxazole (Compound F)

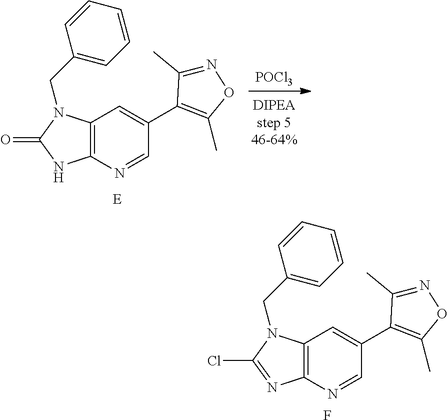

Compound E and phosphorus oxychloride were mixed and then treated with diisopropylethyl amine (DIPEA), which can be added dropwise. The resulting mixture was heated for several hours, cooled, and sampled for reaction completion. If the ratio of Compound E to Compound F was not more than 0.5% then the reaction was complete. Otherwise, the reaction was heated for additional time and sampled as before. After the reaction was complete, the mixture was concentrated then cooled. Ethyl acetate was added and the mixture was concentrated under vacuum several times. Ethyl acetate (EtOAc) was added to the concentrate, the mixture was cooled and then added to aqueous sodium bicarbonate. The organic phase was separated and the organic layer was washed with aqueous sodium bicarbonate and then water. The organic phase was concentrated, ethyl acetate was added, and the mixture was concentrated to assure that the moisture level was not more than 0.2%. The mixture in ethyl acetate was decolorized with carbon. The mixture was concentrated and n-heptane was added. The product was isolated by filtration and dried under vacuum. Drying was complete when residual moisture, ethyl acetate, and n-heptane were not more than 0.5%. ¹H-NMR (MeOH-d₄): δ8.40 (1H), 7.90 (1H), 7.25-7.45 (5H), 5.65 (2H), 2.37 (3H), 2.22 (3H).

### Step F: Synthesis of 1-benzyl-6-(3,5-dimethyl-1,2-oxazol-4-yl)-N-methyl-1H-imidazo[4,5-b]pyridine-2-amine (Compound I)

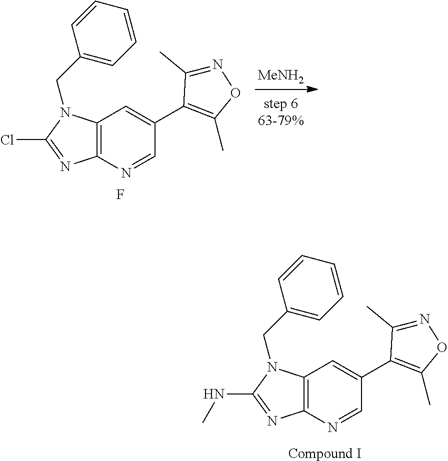

Compound F was mixed with methylamine in tetrahydrofuran (THF) and stirred at ambient temperature until the ratio of Compound F to Compound I was NMT 0.1% by HPLC. After reaction completion, the mixture was concentrated under vacuum, process water added, and the product isolated by filtration. The filter cake was washed with process water. The wet cake was dissolved in hydrochloric acid and the resulting solution was washed with methylene chloride to remove impurities. The aqueous solution was neutralized with a sodium hydroxide solution and Compound I was isolated by filtration, washed with process water, and dried under vacuum. If necessary, to remove any remaining hydrochloric acid, the dried material can be dissolved in ethanol, treated with a solution of sodium hydroxide in ethanol, followed by addition of process water to precipitate the product. Compound I was isolated by filtration, washed with process water, and dried. ¹H-NMR (DMSO-d₆): δ7.96 (d, 1H, J=2.0 Hz), 7.42 (d, 1H, J=2.0 Hz), 7.37 (q, 1H, J=4.2 Hz), 7.32 (m, 2H), 7.26 (m, 1H), 7.24 (m, 2H), 5.30 (s, 2H), 3.00 (d, 3H, 4.5 Hz), 2.34 (s, 3H), 2.16 (s, 3H). ¹³C-NMR (DMSO-d₆): δ164.8, 158.4, 157.7, 156.0, 141.1, 136.4, 128.6 (2C), 127.5, 127.4, 127.2 (2C), 115.8, 114.2 (2C), 44.5, 29.3, 11.2, 10.3.

### Example 2: Crystalline Mesylate of Compound I

About 5 g of Compound I was dissolved in ethanol (115 mL) and a solution of methanesulfonic acid in ethanol (10 mL, 158.7 mg/mL) was added, according to a 1:1 molar ratio. The mixture was shaken at 50° C. for 2 h before concentrated to half volume and stirred overnight. The formed solid (mesylate salt/co-crystal of Compound I Form I) was isolated, dried, and characterized.

The mesylate salt/co crystal of Compound I Form I was also obtained from other solvents and solvent mixtures, including acetone and acetonitrile.

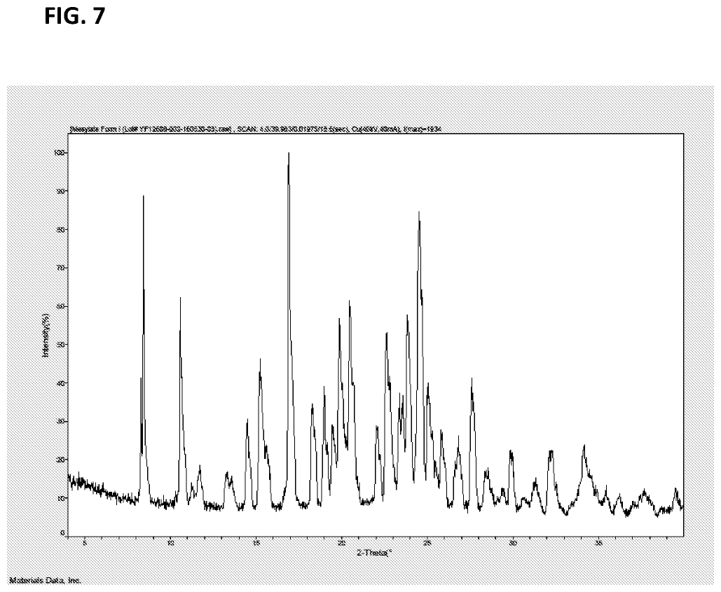

The mesylate salt/co crystal of Compound I Form I was characterized by XRPD comprising the following peaks, in terms of 2-theta, at 8.4±0.2, 10.6±0.2, 11.7±0.2, 14.5±0.2, 15.3±0.2, 16.9±0.2, 18.2±0.2, 19.0±0.2, 19.9±0.2, 20.5±0.2, 22.6±0.2, 23.8±0.2, 24.5±0.2, and 27.6±0.2 degrees, as determined on a diffractometer using Cu-K_{α} radiation tube (FIG. 7).

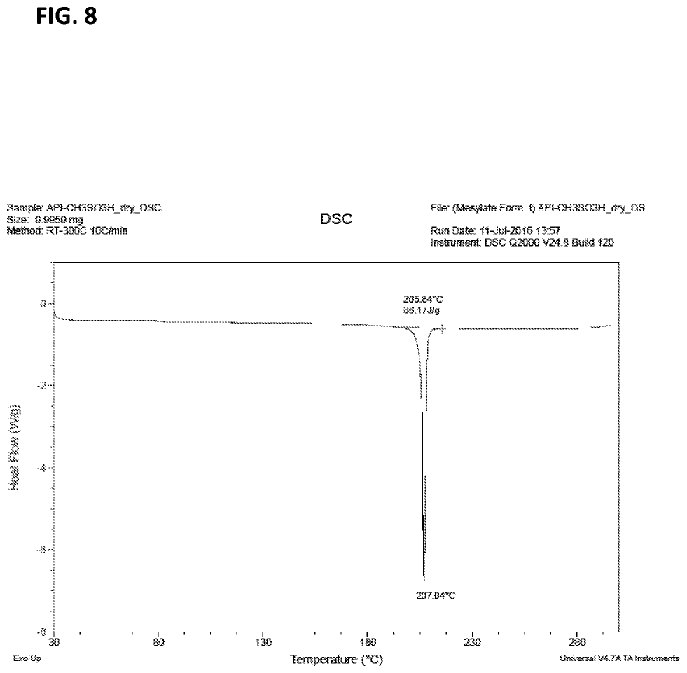

The mesylate salt/co crystal of Compound I Form I was characterized by DSC having an endothermic peak at a temperature of about 207° C. (FIG. 8).

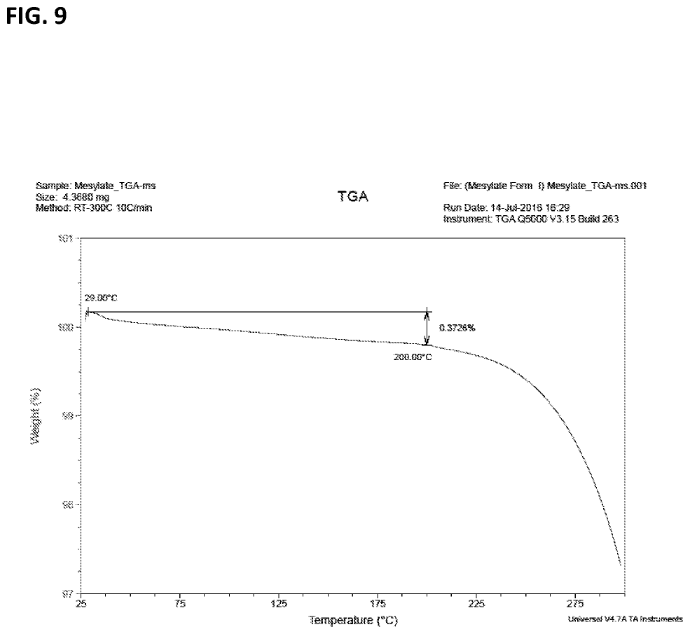

The mesylate salt/co crystal of Compound I Form I was characterized by TGA, having a thermogram as shown in FIG. 9, confirming that Compound I Form I is an anhydrous form.

### Example 3: Synergistic Inhibition of ER+ Breast Cancer Cell Line Viability by Combination of Compound I with Abemaciclib

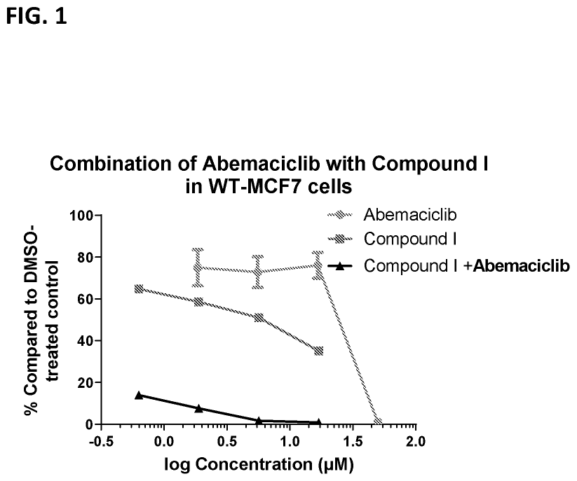

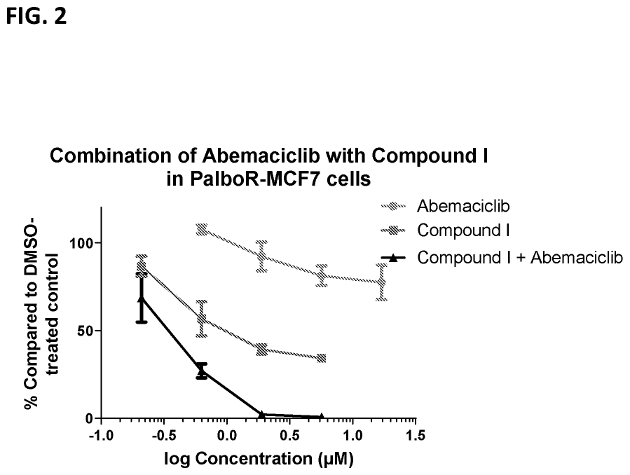

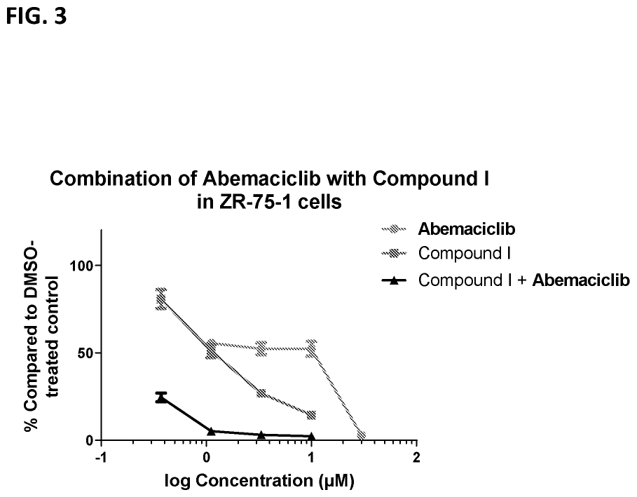

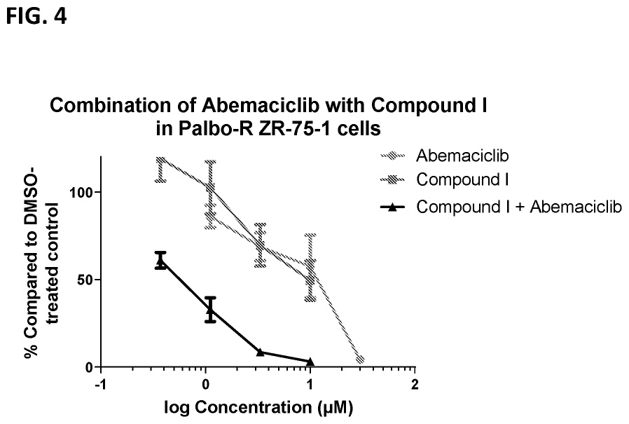

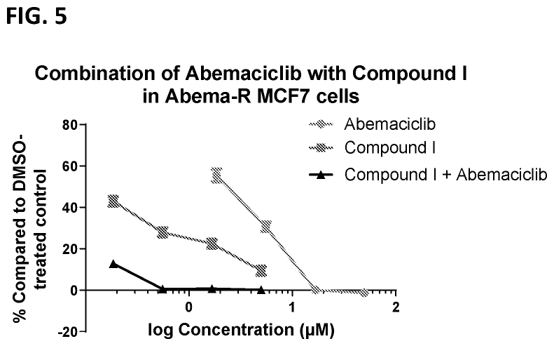

MCF7, Palbo-R-MCF7, ZR-75-1, Abema-R MCF7, and Palbo-R ZR-75-1 cells were plated at a density of 7,500 cells per well in 96 well flat bottom plates in 1640-RPMI media containing 10% FBS and penicillin/streptomycin and incubated for 24 hours at 37° C., 5% CO₂. Media was replaced with 1640-RPM I containing 10% FBS with constant ratios of either Compound I or abemaciclib as single agents, or a combination of both drugs at four different concentrations (2× IC50, 1× IC50, 0.5× IC50, 0.25× IC50), and incubated at 37° C., 5% CO₂ for 7 days. The cells were retreated as described above on the 3rd or 4th day. Triplicate wells were used for each concentration and wells containing only media with 0.1% DMSO were used as a control. To measure cell viability, 100 uL of a 1:100 dilution of GF-AFC substrate into the Assay Buffer (CellTiter Fluor Cell Viability Assay (Promega)) were added to each well and incubated at 37° C., 5% CO₂ for an additional 30-90 minutes. Fluorescence at 380-400 nm Excitation/505 nm Emission was read in a fluorometer and the percentage of cell titer relative to DMSO-treated cells was calculated after correcting for background by subtracting the blank well's signal. IC50 values for single agents were calculated using the GraphPad Prism software. Quantification of synergy was done by calculating combination indices (CI) using the CalcuSyn software (Biosoft) based on the Chou-Talalay algorithm (Chou and Talalay, 1984), and averaging the CI values for the effective doses (ED) 50, 75, and 90. As shown in FIGS. 1-5, addition of Compound Ito abemaciclib resulted in improved inhibition of cell viability compared to either single agent with an average CI value of 0.08-0.35 depending upon the cell line.

### Example 4: Synergistic Inhibition of MCF7 Cell Viability by Combination of Compound I with Fulvestrant

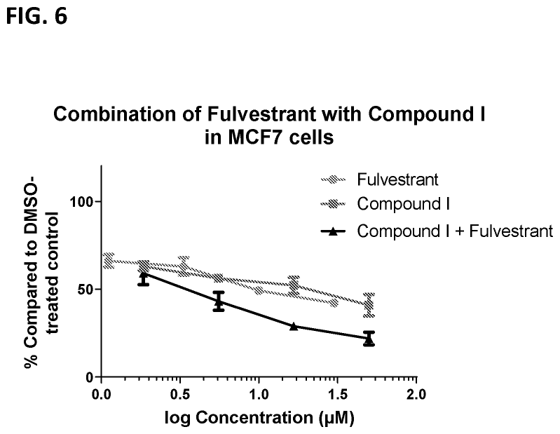

MCF7 cells were plated at a density of 7,500 cells per well in 96 well flat bottom plates in phenol-red free 1640-RPMI media containing 10% charcoal-stripped FBS and penicillin/streptomycin and incubated for 24 hours at 37° C., 5% CO₂. Media was replaced with phenol-red free 1640-RPM I media containing 10% charcoal-stripped FBS and penicillin/streptomycin treated with constant ratios of either Compound I or fulvestrant as single agents, or a combination of both drugs at four different concentrations (2× IC50, 1× IC50, 0.5× IC50, 0.25× IC50), and incubated at 37° C., 5% CO₂ for 7 days. The cells were retreated as described above on the 3rd or 4th day. Triplicate wells were used for each concentration and wells containing only media with 0.1% DMSO were used as a control. To measure cell viability, 100 uL of a 1:100 dilution of GF-AFC substrate into the Assay Buffer (CellTiter Fluor Cell Viability Assay (Promega)) were added to each well and incubated at 37° C., 5% CO₂ for an additional 30-90 minutes. Fluorescence at 380-400 nm Excitation/505 nm Emission was read in a fluorometer and the percentage of cell titer relative to DMSO-treated cells was calculated after correcting for background by subtracting the blank well's signal. IC50 values for single agents were calculated using the GraphPad Prism software. Quantification of synergy was done by calculating combination indices (CI) using the CalcuSyn software (Biosoft) based on the Chou-Talalay algorithm (Chou and Talalay, 1984), and averaging the CI values for the effective doses (ED) 50, 75, and 90. As shown in FIG. 6, addition of Compound I to fulvestrant resulted in improved inhibition of cell viability compared to either single agent with an average CI value of 0.51.

1. A method for treating estrogen receptor positive (ER+) breast cancer comprising administrating to a patient in need thereof a combination of
a. a BET bromodomain inhibitor selected from 1-benzyl-6-(3,5-dimethylisoxazol-4-yl)-N-methyl-1H-imidazo[4,5-b]pyridin-2-amine (Compound I), 1-benzyl-6-(3,5-dimethylisoxazol-4-yl)-1H-imidazo[4,5-b]pyridin-2-amine, and pharmaceutically acceptable salts/co-crystals thereof, and
b. a second therapeutic agent.

2. The method according to claim 1, wherein the BET bromodomain inhibitor is Compound I.

3. The method according to claim 1, wherein the BET bromodomain inhibitor is the mesylate salt/co-crystal of Compound I Form I.

4. The method according to claim 1, wherein the second therapeutic agent is a selective-estrogen receptor degrader (SERD).

5. The method according to claim 1, wherein the second therapeutic agent is a selective-estrogen receptor modulator (SERM).

6. The method according to claim 1, wherein the second therapeutic agent is a selective CDK4/6 inhibitor.

7. The method according to claim 1, wherein the second therapeutic agent is fulvestrant.

8. The method according to claim 1, wherein the second therapeutic agent is palbociclib.

9. The method according to claim 1, wherein the second therapeutic agent is abemaciclib.

10. The method according to claim 1, wherein the second therapeutic agent is ribociclib.

11. The method according to claim 1, wherein the patient previously has been treated with a breast cancer therapy.

12. The method according to claim 1, wherein the patient previously has been treated with a CDK4/6 inhibitor.

13. The method according to claim 1, wherein the patient previously has been treated with chemotherapy.

14. The method according to claim 1, wherein the patient previously has been treated with immunotherapy.

15. The method according to claim 1, wherein the patient is a human.

16. The method according to claim 1, wherein a compound selected from 1-benzyl-6-(3,5-dimethylisoxazol-4-yl)-N-methyl-1H-imidazo[4,5-b]pyridin-2-amine (Compound I) and 1-benzyl-6-(3,5-dimethylisoxazol-4-yl)-1H-imidazo[4,5-b]pyridin-2-amine and pharmaceutically acceptable salts or co-crystals thereof, is dosed with a second therapeutic agent selected from a selective-estrogen receptor degrader (SERD) and a selective CDK4/6 inhibitor without resulting in thrombocytopenia as a dose-limiting toxicity.

17. The method according to claim 16, wherein the second therapeutic agent is fulvestrant, palbociclib, or abemaciclib.
